(19) United States
(12) Patent Application Publication
(10) Pub. No.: US 2014/0128813 A1
(43) Pub. Date: May 8, 2014

(54) CLAMP FOR A CATHETER AND METHODS FOR USE THEREOF

(71) Applicant: Branden D. Rosenhan, Salt Lake City, UT (US)

(72) Inventor: Branden D. Rosenhan, Salt Lake City, UT (US)

(21) Appl. No.: 14/077,202

(22) Filed: Nov. 11, 2013

Related U.S. Application Data
(63) Continuation-in-part of application No. PCT/US2012/036963, filed on May 8, 2012.
(60) Provisional application No. 61/484,117, filed on May 9, 2011.

(51) Int. Cl. A61M 25/02; A61M 25/01

(52) U.S. Cl. CPC A61M 25/02; A61M 25/0111
USPC 604/174

(57) ABSTRACT

A catheter clamp having a generally curved body, a first end, and a second end. Once a central or midline catheter is inserted into a patient's body, the catheter is inserted into the catheter clamp and the clamp is affixed to the patient's skin with, for example, one or more stitches, glue, adhesive bandages, and/or staples. The catheter clamp allows the catheter to adopt a bent profile without risk of kinking the catheter. In addition, when the central line catheter is installed at an interior jugular location, the catheter clamp allows the free ends of the catheter to safely hang down away from the patient's hair for greater patient comfort, ease of access by medical personnel, and reduced danger of infection. The curved profile of the catheter clamp and the catheter also reduces the likelihood that the catheter will be accidentally pulled out or dislodged if the catheter is pulled on.

### CROSS-REFERENCE TO RELATED APPLICATIONS

The present application is a continuation-in-part of PCT/US2012/036963, filed on 9 May 2012, which is a non-provisional and claims the benefit of and priority to U.S. Provisional Pat. App. Ser. No. 61/484,117 filed 9 May 2011 and entitled “CLAMP FOR A CATHETER AND METHODS FOR USE THEREOF,” the entirety of which is incorporated herein by reference.

### BACKGROUND

A central line catheter (“central venous catheter”, “CVC”, “central venous line” or “central venous access catheter”) or midline catheter (a venous catheter placed in similar locations but not terminating in a central vein) are catheters placed into a large vein in the neck (internal jugular vein or external jugular vein), chest (subclavian vein) or groin (femoral vein). Central and midline venous catheters are typically used to administer medication or fluids, obtain blood tests (specifically the “mixed venous oxygen saturation”), and directly obtain cardiovascular measurements such as the central venous pressure. As used herein the term “catheter” may also refer to a tube designed to drain fluid or material within the abdomen, pelvis, chest or other body cavities, such as are use in pulmonology, critical care, general surgery, orthopedic surgery, interventional radiology and other such specialties where catheters are placed with the primary intent to drain material and not infuse material.

In cases of long term infusion or the long term placement of testing equipment, it is typically necessary for the catheter to remain in place for many days. In order to secure such a central line catheter in position at the injection site, the IV tubing is commonly mounted on a thin flexible pad or seat that is sutured to the patient's skin. This combination of tubing and pad comprises a connector to which one or more other IV supply lines having compatible connectors can be attached.

An example of such an anchor is shown in FIGS. 1-3 labeled generally 2. The anchor 2 has a tubular body 4 and a pair of opposed wings 6. Body 4 has a central channel 8. Central channel 8 is typically sized to be the same diameter as the outer diameter of a catheter 14 (e.g., a multi-lumen catheter having multiple access ports) that is to be secured by the anchor 2. Body 4 often has a longitudinal slit 10 that extends entirely through body 4 along the entire length of body 4. The longitudinal slit 10 is typically placed in the body 4 to allow the anchor to be slipped over the catheter 14 after the catheter 14 is installed in the patient's body (e.g., in the external jugular vein).

The wings 6 of the anchor 2 each have an eyelet 12. In use, a catheter 14 is placed through slit 10 into channel 8. Because of the tight tolerance in the diameter of central channel 8 and the outer diameter of catheter 14, it is very difficult if not impossible to thread catheter 14 through channel 8. Thereafter, anchor 2 is moved to the desired position. Wings 6 are pinched together toward slit 10. A suture 16 is placed through eyelets 12 and tissue into the patient's tissue (not shown) to secure the catheter 14 and the anchor 2 to the patient's tissue.

When the central line catheter is positioned in the jugular vein using the anchor 2 described above, the internal end of the catheter enters the neck of the patient and the external end exits the neck and extends toward the patient's head. A number of problems, however, have arisen with respect to such placement. For example, the location of the access ports can be uncomfortable for the patient and inconvenient for medical personnel to access. In addition, the access ports can extend into the patient's hair and ear, which is a potential route for contamination or infection of the central line. If the patient is of shorter stature, more of the catheter will protrude out of the body and securing the catheter may require suturing or stapling at an inconvenient site very near to or directly under the patient's upper neck, jaw, or ear. Additionally, this current method does little to address the potential for infection at the site where the catheter is inserted under the skin.

### BRIEF SUMMARY

The present disclosure relates to a catheter clamp that has a generally curved body. Once a central line catheter, such as a multi-lumen central line catheter, is inserted into a patient's body (e.g., at an interior jugular location), the catheter is inserted into the catheter clamp and the clamp is affixed to the patient's skin with, for example, one or more sutures, staples, or other suitable affixing mechanisms. The catheter clamp described herein includes a curved body that allows a catheter placed in the curved body to adopt a bent profile that redirects the free end of the catheter away from the direction of insertion, which, for example, reduces the likelihood that the catheter will be accidentally pulled out or dislodged if the free end of the catheter is pulled on. Likewise, the curved body of the catheter clamp supports the bent profile to greatly reduce risk of kinking the catheter. In addition, when the central line catheter is installed at an interior jugular location, the catheter clamp allows the free end of the catheter to safely hang down away from the patient's hair and ear for greater patient comfort, ease of access by medical personnel, and reduced danger of infection. Such positioning will also be advantageous to patients, allowing them to freely move without discomfort or limitation. Likewise, when laying in a hospital bed the likelihood of patients rolling over onto the catheter ports when turning will be greatly reduced.

In one embodiment, a catheter clamp is described. The catheter clamp includes a curved body extending between a first end and a second end, a channel formed in the curved body into which a catheter tube can be inserted, and at least one eyelet coupled to the curved body for affixing the catheter clamp to the skin. The curved body is shaped to permit a free end of the catheter tube to be oriented away from a catheter insertion site and to support a bent profile of the catheter tube to reduce risk of kinking the catheter tube.

In one embodiment, the curved body defines an angle in a range between about 90° and about 210°, or any angle therebetween. For example, the curved body may define an angle of about 90°, 95°, 100°, 105°, 110°, 115°, 120°, 125°, 130°, 135°, 140°, 145°, 150°, 155°, 160°, 165°, 170°, 175°, 180°, 185°, 190°, 195°, 200°, 205°, or 210°. Preferably, the curved body defines an angle of about 135° or 180°.

In one embodiment, the curved body defines a radius of about 1.5 to about 3 cm, but may be as long as 5 cm to accommodate larger catheters (e.g., dialysis catheters). However, it will be understood that the curved body can be larger or smaller depending on the diameter of the catheter to be affixed to the body. For example, the curved body and the channel formed therein can be configured to accommodate a catheter having a size in a range from about 5 French to about 15 French, or about 7 French to about 9 French. In another example, the channel can be sized with an inner radius of curvature sized to accommodate a catheter having a size in a range from about 5 French to about 7 French and an outer radius of curvature sized to accommodate a catheter having a size in a range from about 9 French to about 11 French. In yet another example, the curved body includes a pliable material in the channel configured to accommodate and retain catheters having sizes ranging from about 5 French to about 15 French.

In one embodiment, a method for securing a central line catheter to a patient's skin is described. The method includes (1) positioning a catheter in a body lumen or a body cavity of a patient, (2) positioning a free end of the catheter in a catheter clamp as described herein, and (3) securing the catheter clamp to the patient's skin.

In yet another embodiment, a kit is described. The kit may include but is not limited to, a catheter, apparatus for inserting the catheter into a body lumen or a body cavity of a patient, and a catheter clamp as described herein.

The apparatus for inserting the catheter may include one or more of a procedure tray, an anesthetic, a sterilizer for the patient's skin, a needle and suture or staples, a guide wire, a hollow or ‘cook’ needle for central venous puncture, a dilator, and a scalpel.

In one embodiment, the kit further includes a suture needle and a length of suture, a surgical stapler, and/or one or more other suitable mechanisms for affixing the catheter to the patient's skin.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the embodiments of the invention will be rendered by reference to specific embodiments thereof which are illustrated in the appended drawings. It is appreciated that these drawings depict only illustrated embodiments of the invention and are therefore not to be considered limiting of its scope. The embodiments of the invention will be described and explained with additional specificity and detail through the use of the accompanying drawings in which:

FIG. 1 illustrates a top view of a prior art catheter clamp.

FIG. 2 illustrates an end on view of the catheter clamp of FIG. 1.

FIG. 3 illustrates the catheter clamp of FIG. 1 in use with a catheter.

FIG. 4A illustrates a top view of a catheter clamp having a curved body, according to one embodiment of the present invention.

FIG. 4B illustrates a bottom view of the catheter clamp of FIG. 4A, according to one embodiment of the present invention.

FIG. 4C illustrates a cut-away view of the catheter clamp of FIG. 4B along line 4C-4C, according to one embodiment of the present invention.

FIG. 4D illustrates a perspective view of the catheter clamp of FIG. 4A, according to one embodiment of the present invention.

FIG. 4E illustrates a cut-away view of a catheter clamp that includes a pliable member and a rigid member, according to one embodiment of the present invention.

FIG. 4F illustrates a cut-away view of a catheter clamp that includes a pliable member and a rigid member, according to one embodiment of the present invention.

FIG. 5 illustrates a top view of the catheter clamp of FIG. 4A with an associated catheter, according to one embodiment of the present invention.

FIG. 6 illustrates a top view of a catheter clamp with an associated catheter, according to one embodiment of the present invention.

FIG. 7A illustrates a perspective view of a catheter clamp having a domed cap positioned to cover a site where a catheter is inserted into a patient's body, according to one embodiment of the present invention.

FIG. 7B illustrates an underside view of the catheter clamp illustrated in FIG. 7A, according to one embodiment of the present invention.

FIG. 7C illustrates a top view of the catheter clamp illustrated in FIG. 7A with an associated catheter, according to one embodiment of the present invention.

FIG. 7D illustrates a cut-away view of the catheter clamp of FIG. 7A showing a portion of a catheter as it enters a patient's body and also showing the domed cap shielding catheter insertion site and the catheter adjacent to the insertion site, according to one embodiment of the present invention.

FIGS. 8A-8B illustrate an expandable catheter clamp that can be expanded in order to adjust to the size of the patient's external anatomy and also for increasing/decreasing length of catheter inside the patient's anatomy, according to one embodiment of the present invention.

FIG. 9 illustrates a catheter clamp having rounded first and second ends to protect the catheter from kinking or bending where the catheter enters or exits the first and second ends, according to one embodiment of the present invention.

FIG. 10 illustrates a catheter clamp having two potential sites of exit which allows the operator to choose the desired direction and ultimate resting location of the catheter access ports, according to one embodiment of the present invention.

FIG. 11 illustrates a catheter clamp that can be repositioned into a desired angle and secured to a patient's skin, which allows an operator to choose the desired direction and ultimate resting location of the catheter access ports while also protecting the catheter from kinking, according to one embodiment of the present invention.

FIG. 12A illustrates a perspective view of a shapeable catheter clamp that includes a number of interconnected open rings, according to one embodiment of the present invention.

FIG. 12B illustrates a view of the catheter clamp of FIG. 12A in a curved orientation, according to one embodiment of the present invention.

FIG. 13A illustrates a perspective view catheter clamp having a curved body that includes a channel into which a catheter can be placed and a lid to seal the channel, according to one embodiment of the present invention.

FIG. 13B illustrates a perspective view of the catheter clamp illustrated in FIG. 13A with the lid closed, according to one embodiment of the present invention.

FIG. 14 illustrates a kit for deploying a catheter into a patient's anatomy and a catheter clamp for affixing the catheter to the patient's skin, according to one embodiment of the present invention.

### DETAILED DESCRIPTION

The present disclosure relates to a catheter clamp that has a generally curved body. Once a central line catheter, such as a multi-lumen central line catheter, or drainage catheter is inserted into a patient's body (e.g., at an interior jugular location), the catheter is inserted into the catheter clamp and the clamp is affixed to the patient's skin with, for example, one or more sutures, staples, glue, or other suitable affixing mechanisms. The catheter clamp described herein includes a curved body that allows a catheter placed in the curved body to adopt a bent profile that redirects the free end of the catheter away from the direction of insertion, which, for example, reduces the likelihood that the catheter will be accidentally pulled out or dislodged if the free end of the catheter is pulled. Likewise, the curved body of the catheter clamp supports the bent profile to greatly reduce risk of kinking the catheter. In addition, when the central line catheter is installed at an interior jugular location, the catheter clamp allows the free end of the catheter to safely hang down away from the patient's hair and ear for greater patient comfort, ease of access by medical personnel, and reduced danger of infection.

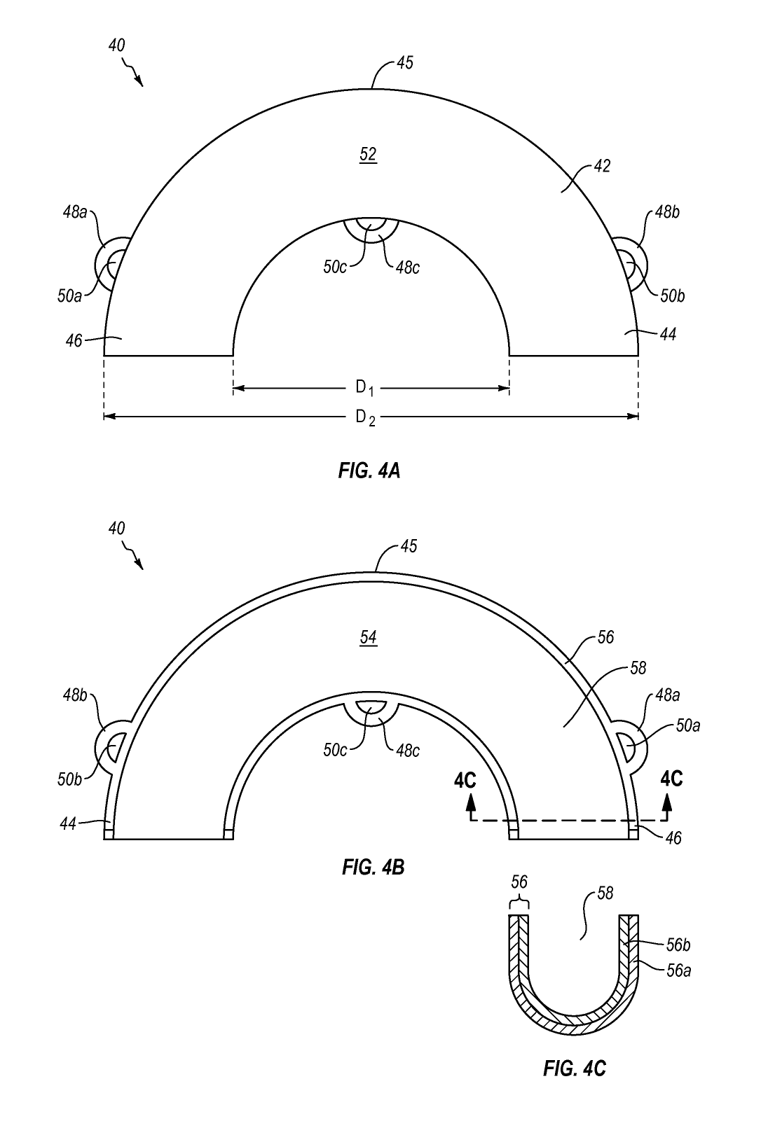

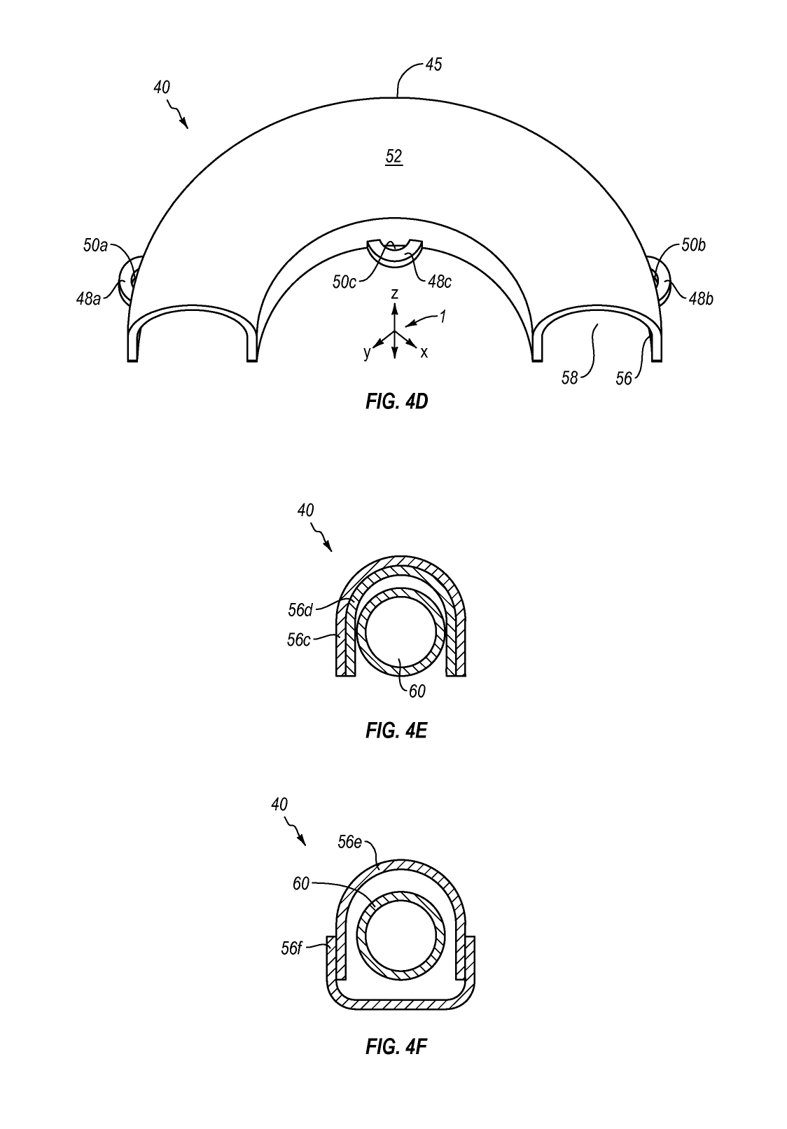

Referring now to FIG. 4A, a top view 52 of a catheter clamp 40 according to one embodiment of the invention is illustrated. The catheter clamp 40 includes a curved body 42 having a first end 44 and a second end 46. In the illustrated embodiment, the first and second ends 44 and 46 are shown terminating at similar (i.e., mirrored) locations. In other embodiments (not shown), the first and second ends 44 and 46 can have different termini. For example, the curved body 42 of the catheter clamp 40 in the illustrated embodiment defines an angle of about 180° with the termini essentially ending on a hemispheric line of a circle. In one embodiment, one of the termini could extend below the hemispheric line. In such an embodiment, the extended terminus could continue around the circle so that the curved body 42 defines an angle greater than 180°, or the extended terminus could extend down without continuing the angle of the curved body (e.g., the extended terminus may hang essentially straight below the hemispheric line). In one embodiment, one of the termini could be terminated above the hemispheric line such that the curved body 42 defines an angle of less than 180°.

Also in the illustrated embodiment, the first and second ends 44 and 46 are shown as being within the same plane as the rest of the body 42. In other embodiments (not shown), the first and second ends 44 and 46 can be non-planar with termini that extend above or below the plane of the body 42. For example, the anatomies that the catheter clamp can be attached to are not necessarily planar. As such, the body 42 can be shaped with the first and second ends 44 and 46 extending above or below the plane of the body 42 to accommodate different anatomies. In other words, catheter clamp 40 may be designed to generally lie against a patient's neck, chest, leg or other attachment sites. Other attachment sites might include but are not limited to arm, shoulder and groin. For example, the catheter clamp 40 may be curved in a z-axis direction (see, e.g., coordinate system 1) about the y-axis to generally match a range of attachment site geometries.

In the illustrated embodiment, the catheter clamp 40 forms an approximately 180° bend. In other embodiments (not shown), the angle formed by the catheter clamp 40 can range from about 90° to about 210°. For example, the curved body may define an angle of about 90°, 95°, 100°, 105°, 110°, 115°, 120°, 125°, 130°, 135°, 140°, 145°, 150°, 155°, 160°, 165°, 170°, 175°, 180°, 185°, 190°, 195°, 200°, 205°, or 210°. Preferably, the curved body defines an angle of about 135° or about 180°. It should be noted that such a catheter clamp might be constructed of pliable materials with the ability to form various angles based on a predetermined or desired configuration. For example, one such catheter clamp may have a predetermined configuration of approximately 135°, but have the ability to be deflected and sutured into place having a final defined angle of either 90°, 180°, or any angle therebetween.

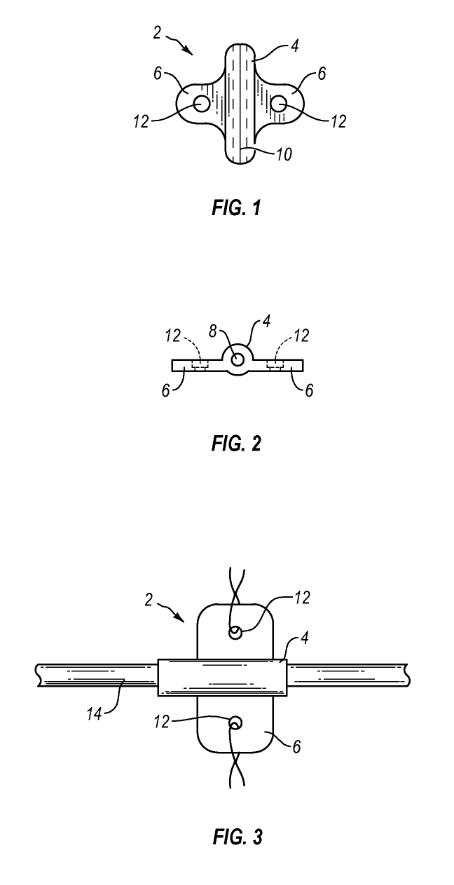

In addition to moving the catheter's position away from, for example, the patient's hair, etc, the bend may alter the forces applied to the catheter at the insertion site. For example, forces applied to one end of a straight catheter clamp (see, e.g., FIGS. 1-3) are applied in the same direction as the insertion of the catheter. Providing a bend changes the vectors of the forces applied to the free end of the catheter with respect to the inserted end. For example, because the free end of a catheter secured in the curved body 42 of the catheter clamp 40 is directed away from an insertion point, a pulling force applied to the free end of the catheter will be considerably less likely to dislodge or pull out the catheter. This may reduce the risk of accidental withdrawal of the catheter from the patient. This is in direct contrast with the prior art device illustrated in FIGS. 1-3 or other such devices (not shown) as currently used in surgical or interventional securement. Such known devices do not direct the catheter vector generally 180° or another angle away from the catheter skin exit site but rather secure catheters without significantly changing the potential dislodging vector of force.

In one example, the catheter clamp can be co-extruded from more than one type of plastic such that the apex area 45 includes a soft, moldable plastic that allows the curved body 42 to be shaped. In contrast, other portions can be made from a stiffer plastic that can better anchor the catheter in the patient's vein. For example, the areas near the apex 45 and/or the first and second ends 44, 46 may include a softer material than the material used to form the rest of the catheter clamp 40. Varying the hardness/pliability of the materials used to form the catheter clamp 40 at these or other locations may help to prevent kinking of the catheter while in use. Likewise, selected positioning of hard and soft materials may allow the catheter clamp 40 to better support a catheter tube secured by the clamp 40, further helping to prevent kinking of the catheter tube while in use. Such materials may also be substantially clear, allowing visualization of the catheter body.

The catheter clamp 40 further includes tabs 48a-48c with eyelets 50a-50c for attaching the catheter clamp 40 to the patient's skin. These tabs, 48a-48c, are illustrated in FIGS. 4A, 4B, and 4D. The catheter clamp can be attached to the patient's skin, for example, with sutures or staples. In another embodiment, the catheter clamp 40 can be attached to the patient's skin with an adhesive. The adhesive can be used alone to attach that catheter clamp to the patient's skin or the adhesive can be used together with the eyelets 50a-50c and one or more sutures or staples. In one example, sutures can be looped through each of the eyelets 50a-50c into the patient's skin to secure the catheter clamp 40. In another example, one length of suture can be run through each of the eyelets 50a-50c into the patient's skin to secure the catheter clamp 40.

The curved body 42 includes an inner diameter d1 and an outer diameter d2. Diameters d1 and d2 can be selected to allow the catheter clamp 40 to accommodate catheters having a wide variety of diameters (e.g., 5 French to 15 French). In one embodiment, the radii of curvature defined by d1 and d2 can be different to allow the catheter clamp to accommodate a wider range of catheter diameters. For example, the inner diameter, d1, can be sized to accommodate catheter sizes in a range from about 5-7 French and the outer diameter, d2, can be sized to accommodate catheter sizes in a range from about 9-11 French.

Referring now to FIGS. 4B and 4D, the underside (indicated at 54 in FIG. 4B) and a perspective view of the catheter clamp are illustrated. In the underside and perspective views, the channel 58 that is formed in the body 42 to accommodate the catheter is clearly visible. The channel 58 runs the length of the curved body 42 and is defined by a lateral channel wall 56. As with the diameters d1 and d2, the channel can be sized and shaped to accommodate catheters having a wide variety of diameters (e.g., 5 French to 15 French).

Referring to FIG. 4C, an embodiment of the channel 58 is illustrated. In the embodiment of FIG. 4C, the lateral channel wall 56 of channel 58 includes a soft inner material 56b and a rigid outer material 56a. The soft inner material 56b is selected to allow the channel 58 to accommodate a wider variety of catheter sizes while the rigid outer material 56a is designed to allow the catheter clamp 40 to provide support to the catheter disposed in the channel 58. Referring to FIGS. 4E and 4F, embodiments of catheter clamps 40 that include a pliable member 56d and 56f and a rigid member 56c and 56e are shown. A catheter tube 60 is illustrated in FIGS. 4E and 4F for reference. In FIGS. 4E and 4F, the pliable member 56d and 56f is disposed over the catheter tube 60 and the rigid member 56c and 56e is disposed over the pliable member 56d and 56f. Such a system can, for example, allow the catheter clamp 40 to more securely engage the catheter tube 60 and can allow the catheter clamp 40 to accommodate a wider range of catheter tube sizes. The location, length, thicknesses, hardness, or other characteristics of the materials may be varied to accommodate various sizes of catheters while preventing kinking of the catheters.

Infections are one of the most common complications associated with installation of a central line catheter. In order to help prevent infection, one or more portions of the catheter clamp 40 can include one or more anti-microbial substances such as, but not limited to, benzalkonium chloride or silver compounds. For example, one or more portions of the catheter clamp 40 can be coated or impregnated with one or more anti-microbial substances. In another example, the pliable member (e.g., 56b, 56d, or 56e) may be comprised of an absorbent material that can absorb and retain a one or more anti-microbial substances, such as a liquid antimicrobial substance.

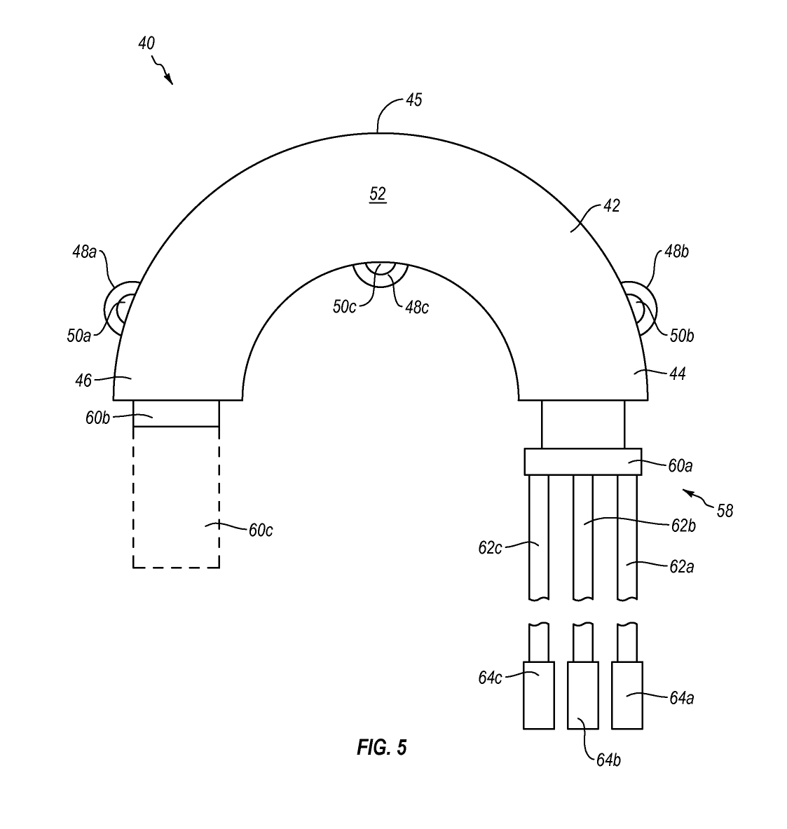

Referring now to FIG. 5, the catheter clamp 40 of FIG. 4A is shown with a catheter 58 disposed in the curved body 42. In the illustrated embodiment, the catheter 58 is a multi-lumen catheter. However, it should be understood that other types of catheters can be secured by the catheter clamp 40.

The catheter 58 includes a first end 60a, a second end 60b, and a portion 60c that extends under the patient's skin. Extending from the first end 60a are a number of lumens 62a-62c. Each lumen 62a-62c includes a separate hub 64a-64c. The hubs 64a-64c can, for example, be Luer lock hubs that can be coupled with a syringe or another device for injection or withdrawal of substances through a selected lumen of the catheter 58. Likewise, one or all of the hubs 64a-64c can be sealed when they are not being used to prevent blood or fluid loss from the patient and/or to prevent infection.

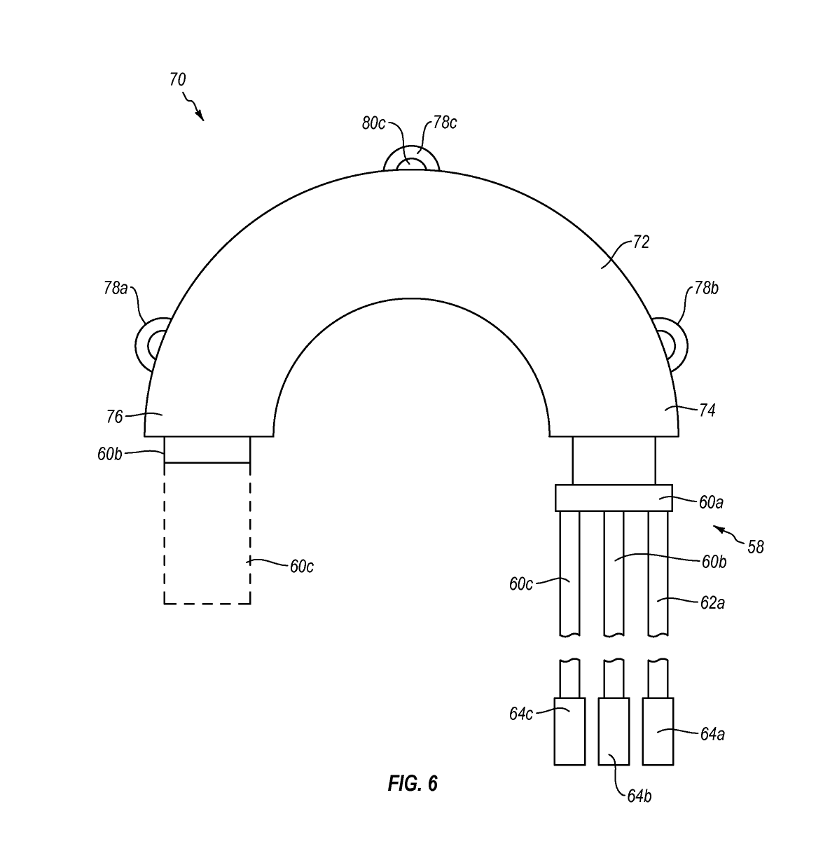

Referring now to FIG. 6, another embodiment of a catheter clamp 70 is illustrated. The catheter clamp 70 includes a first end 74, a second end 76, and a number of suture tabs 78a-78c positioned between the first and second ends 74 and 76. Catheter clamp 70 is similar in most respects to catheter clamp 40, except the position of the tabs 78a-78c is altered. Changing the position of the tabs (see, e.g., 48a-48c versus 78a-78c) can allow different patterns of sutures or staples for securing the catheter clamp to a patient's skin. For instance, some patterns of suture tabs may be better in some applications than in others. It should be noted that FIGS. 4a-6 illustrate examples of positioning for the suture tabs and that other arrangements are possible under the scope of the present disclosure. The positioning of the suture tabs may facilitate the alteration of the force vectors applied by pulling the free end of the catheter with respect to the inserted end, thus preventing or reducing the risk of accidental withdrawal.

The catheter clamp 70 is also shown with a catheter 58 disposed in the curved body 72. The catheter 58 includes a first end 60a, a second end 60b, and a portion 60c that extends under the patient's skin. Extending from the first end 60a are a number of lumens 62a-62c.

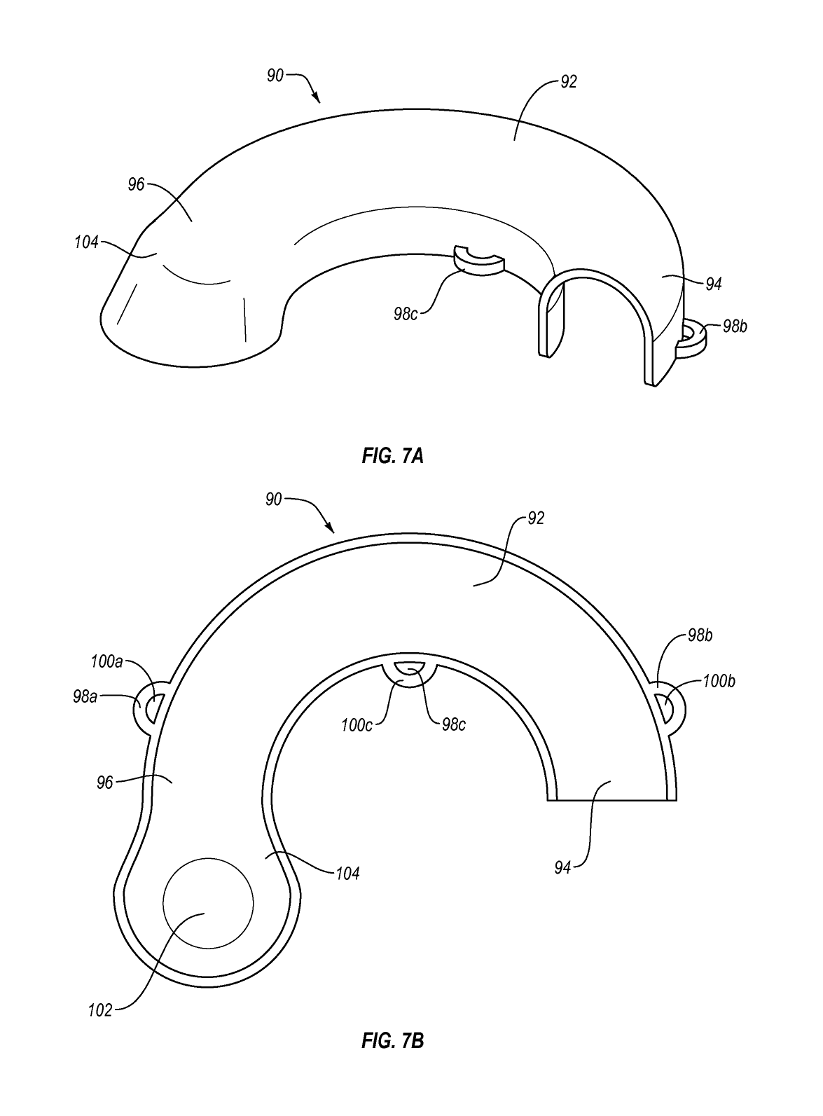

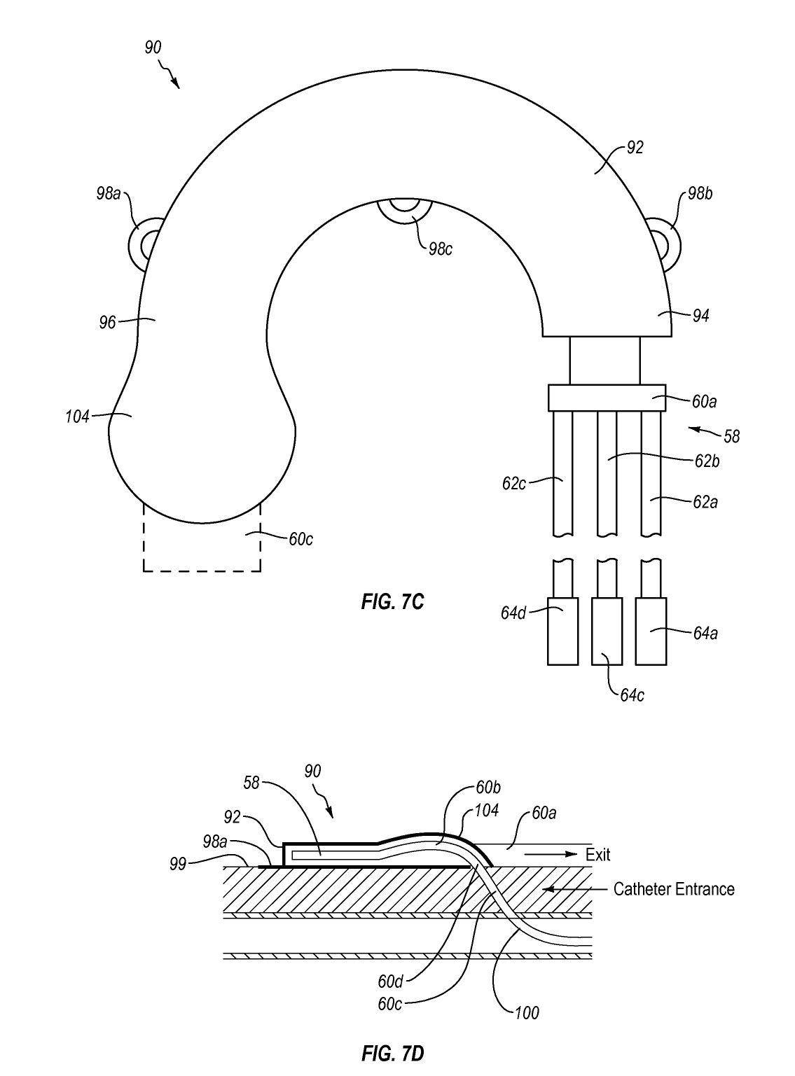

Referring to FIGS. 7A-7D, yet another example of a catheter clamp 90 is illustrated, according to one embodiment of the present invention. FIG. 7A illustrates a perspective view of the catheter clamp 90 and FIG. 7B illustrates a view of the underside of the catheter clamp 90. The catheter clamp 90 includes a first end 94, a second end 96, and a number of suture tabs 98a-98c positioned between the first and second ends 94 and 96. Catheter clamp 90 is similar in most respects to catheter clamp 40, except catheter clamp 90 includes a domed cap 104 at the second end of the clamp 90. The domed cap 104 is located and sized to be positioned over a puncture site (e.g., a venous puncture site or chest tube or drainage catheter site) where a catheter is inserted into a patient's body. In conventional applications, the puncture site is particularly prone to infection because there tends to be slight amounts of movement of the catheter in and out of the body at the puncture site. This creates an avenue for infectious agents to enter the patient's body. It is difficult to protect this area with commonly used linear catheter clamps, such as the catheter clamp 2 shown in FIGS. 1-3.

The domed cap 104 protects this area by substantially (or even completely) covering the site where the catheter enters the body. In addition, the domed cap 104 can include an absorbent patch 102 or the like that may be soaked or impregnated with an antimicrobial substance such as, but not limited to, benzalkonium chloride, a silver compound, an antibiotic, or the like to further protect the area around the puncture site.

Referring now to FIG. 7C, the catheter clamp 90 is also shown with a catheter 58 disposed in the curved body 72. The catheter 58 includes a first end 60a, a second end, and a portion 60c that extends under the patient's skin. In the view illustrated in FIG. 7C, the second end of the catheter 58 cannot be seen because it is covered by the dome 104. Extending from the first end 60a are a number of lumens 62a-62c.

Referring now to FIG. 7D, a partial cut-away view of the catheter clamp 90 and the catheter 58 with the portion 60c that extends under the patient's skin 99 and extending into a blood vessel 100. As can be seen in FIG. 7D, the domed cap 104 protects the site of entry 60d where the end portion of the catheter 60c passes through the patient's skin 99. As discussed above, the site of entry 60d is particularly prone to infection and the domed cap 104 protects the site of entry 60d and the associated tissue and blood vessels from contamination with foreign matter, bacteria, and the like.

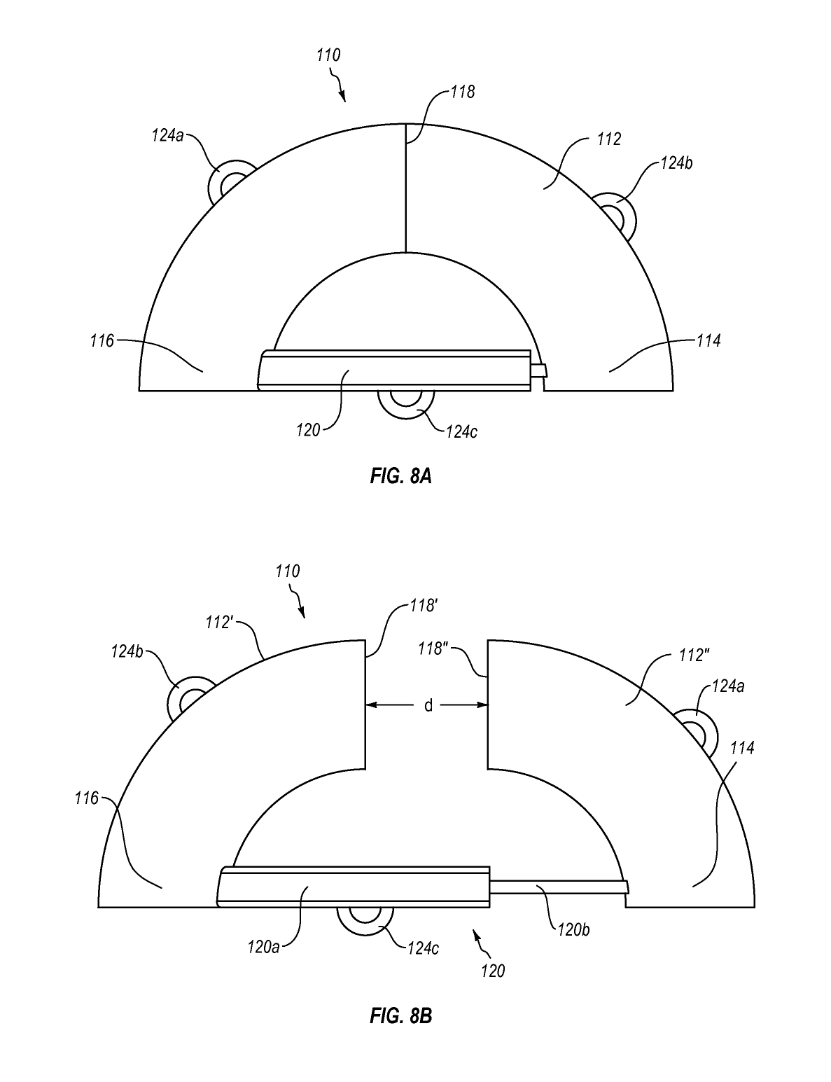

Referring now to FIGS. 8A and 8B, an expandable catheter clamp 110 is illustrated. The expandable catheter clamp 110 includes a first end 114, a second end 116, and a number of suture tabs 124a-124c positioned between the first and second ends 114 and 116. In addition, the expandable catheter clamp 110 includes a split 118 and a slidable expansion member 120 that allows the expandable catheter clamp 110 to be expanded in order to adjust to the size of the patient's external anatomy (e.g., different sized neck jugular areas) and also for increasing/decreasing length of catheter inside the patient's anatomy.

As can be better seen in FIG. 8B, the slidable expansion member 120 of the expandable catheter clamp 110 includes a first member 120a and a second member 120b that can slide in and out of the first member 120a to allow the catheter clamp 110 to be expanded or contracted. As illustrated in FIG. 8B, when the expandable catheter clamp 110 is in an expanded state, the split 118 separates into split 118′ and 118″. Likewise, the curved body 112 separates into a first half 112′ and a second half 112″.

The expandable catheter clamp may be additionally equipped with a selection of clamp covers (not shown) to cover the portion of the catheter that is exposed when the catheter clamp is in one or more expanded states. Additionally, the expandable catheter clamp may include an integral member adapted to keep the catheter covered as the expandable clamp is expanded or contracted.

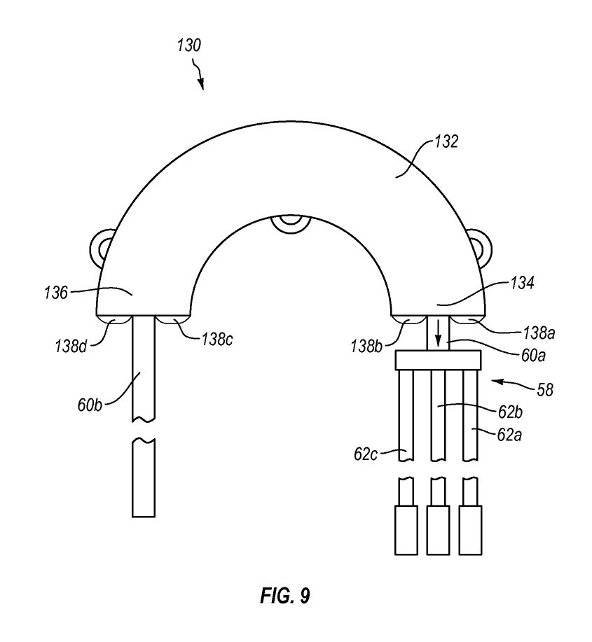

Referring now to FIG. 9, a catheter clamp 130 having rounded ends to better support a catheter clamped in the catheter clamp 130. The catheter clamp 130 includes a first end 134, a second end 136, and a number of rounded bumpers 138a-138d positioned at the first and second ends 134 and 136. The catheter clamp 130 is also shown with a catheter 58 disposed in the curved body 132. The catheter 58 includes a first end 60a, a second end 60b, and a portion 60c that extends under the patient's skin. Extending from the first end 60a are a number of lumens 62a-62c.

The rounded bumpers 138a-138d may be adapted to protect the catheter 58 from kinking or bending where the catheter enters or exits the first and/or second ends 134 and 136. The rounded bumpers 138a-138d may be relatively harder or relatively softer than the body 132 of the catheter clamp 130 depending on the particular application.

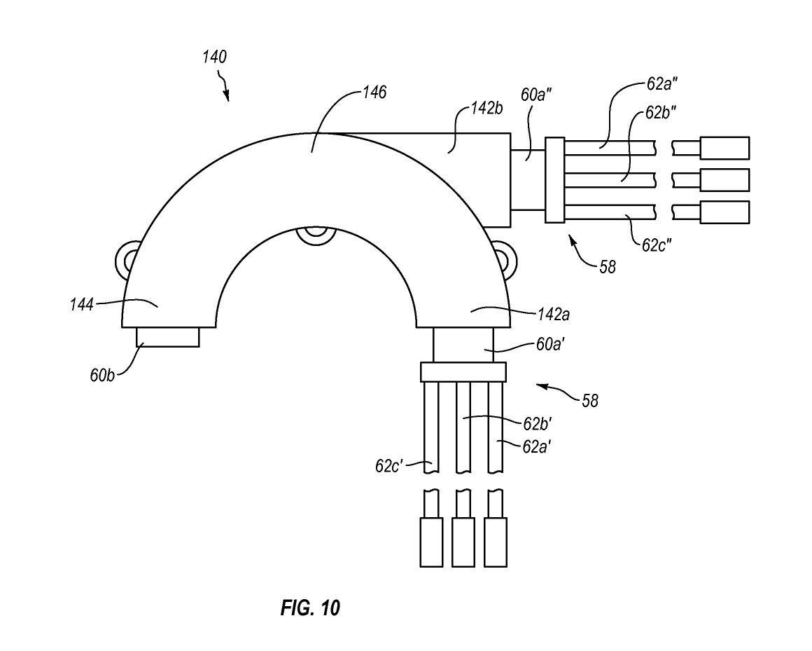

Referring now to FIG. 10, a catheter clamp 140 having multiple catheter paths is illustrated. The catheter clamp 140 includes a second end 144 and one first end 142a and a second first end 142b. The catheter clamp 140 may contain two or more concurrent combinations of first ends (e.g., 142a and 142b) that are form angles between the second end 144 and the first ends of about from about 90° to about 210°. Likewise, FIG. 10 may represent two different end securements of a flexible or pliable catheter of a single catheter path and preferred angle of 135° that may be secured in either the 142a position or the 142b position, depending on the desire of the clinician based on patient/care factors such as but not limited to anatomy, wounds, skin thickness, etc.

In the illustrated embodiment, the catheter clamp 140 has a catheter 58 disposed in the curved body 146. The catheter 58 is similar to the catheters described in reference to the other Figures herein. However, for the purpose of illustration and not limitation, the catheter is shown with a second end 60b and alternate first ends 60a′ and 60a″. In the first example, the catheter 58 disposed in the curved body 146 with the first end 60a′ position to form and angle of approximately 180° between the first end 60a′ and the second end 60b. In the second example, the catheter 58 disposed in the curved body 146 with the first end 60a″ position to form and angle of approximately 90° between the first end 60a″ and the second end 60b.

Such embodiments provide for a first, second and third (fourth, etc) end that may allow unique configurations not accounted for in current devices. In the illustrated embodiment, the ends of the clamp 140 are both generally within the same plane (i.e. the same plane as the page). In other embodiments, the ends may be in different planes.

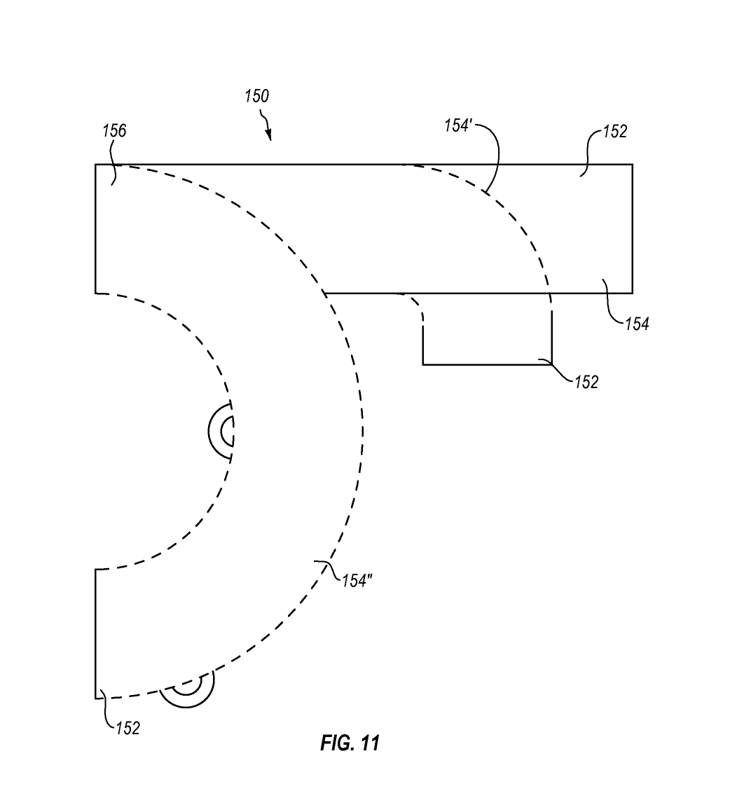

Referring now to FIG. 11, a catheter clamp 150 that includes a shapeable body 152 is shown. In addition to providing shapeable body, the catheter clamp can protect a catheter secured in the clamp from kinking. The catheter clamp 150 includes a body 152 having a first end 154 and a second end 156. The body 152 can be bent to allow an operator to choose the desired direction and ultimate resting location of the catheter access ports.

For example, as illustrated in a first view, the catheter clamp 150 can be used with a straight body 154, with the body bent approximately 90° 154′, and/or with the body bent approximately 180° 154″, or any angle therebetween. In one embodiment, the shapeable catheter clamp may be made from multiple materials, such as moldable plastic with a more rigid spine being made from a metal or a similar ductile material that can be readily bent and shaped by an operator. A multiplicity of materials will serve to maximize the ability of the clamp to direct the catheter in the desired direction while conforming appropriately to the patient anatomy.

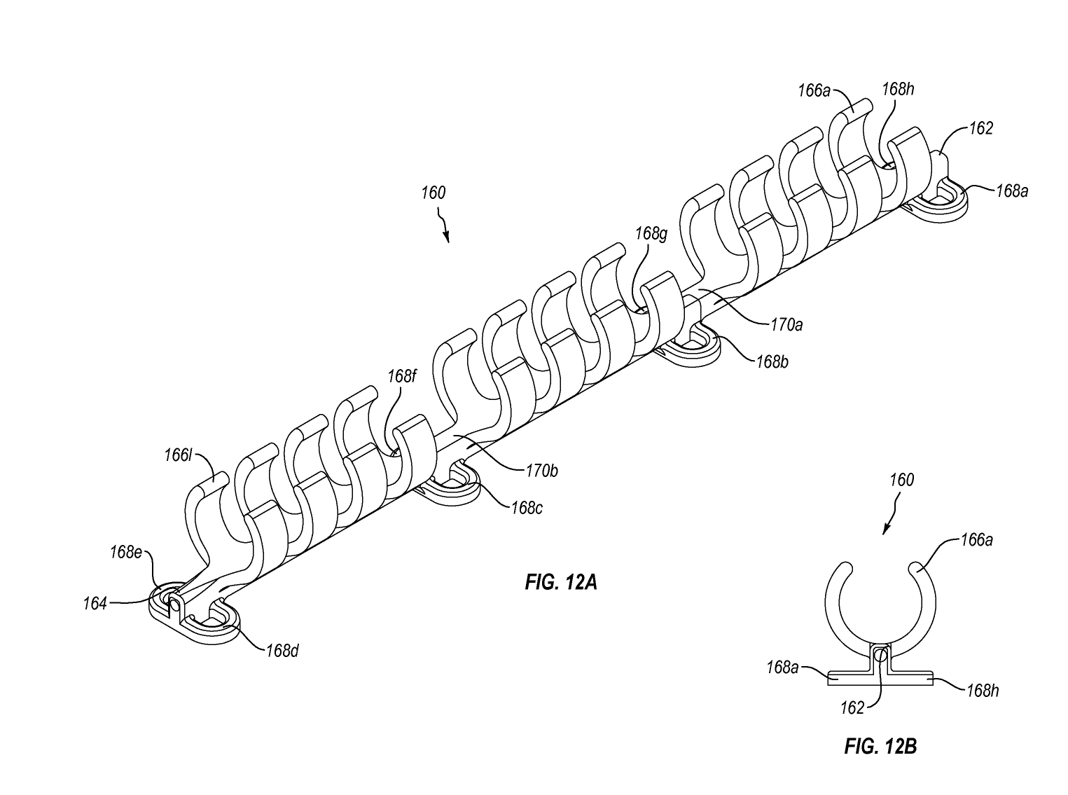

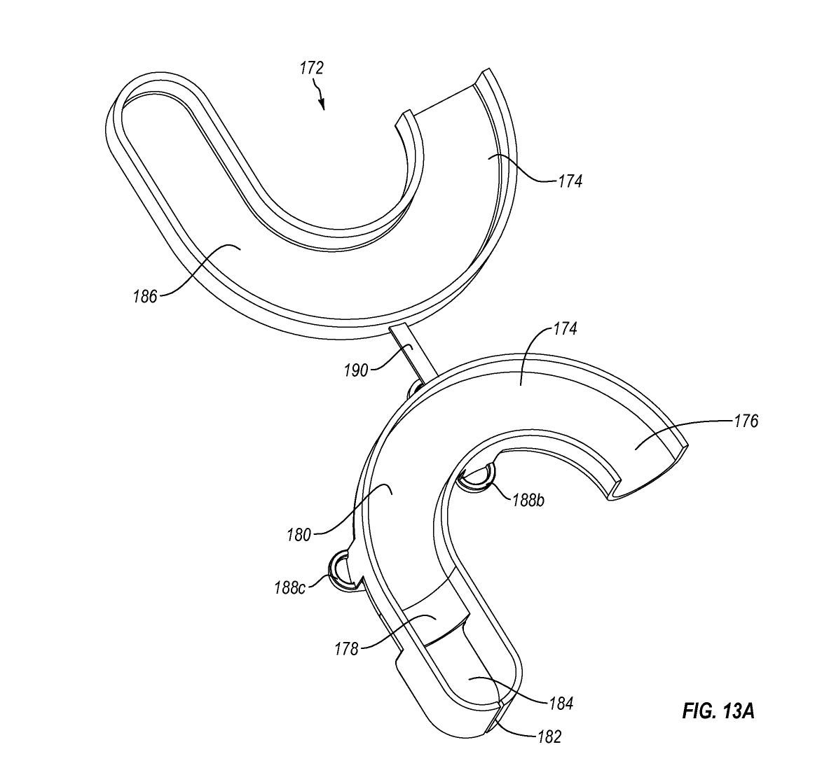

Referring now to FIGS. 12A and 12B, another embodiment of a shapeable catheter clamp 160 is illustrated. The catheter clamp 160 includes a number of open ring structures 166a-166l extending between a first end 162 and a second end 164. The catheter clamp 160 also includes a number of suture tabs 168a-168h that can be used to affix the catheter clamp to a patient skin.

The open ring structures 166a-166l are interconnected by a central spine 172 that also extends between the first end 162 and the second end 164. The central spine 172 includes a number of open sections 170a and 170b between the ring structures that may facilitate shaping the catheter clamp 160. The central spine 172 may be made from a moldable plastic with a more rigid internal spine (not shown) being made from a metal or a similar ductile material that can be readily bent and shaped by an operator. Shaping the catheter clamp 160 maximizes the ability of the clamp 160 to direct the catheter in the desired direction while conforming appropriately to the patient's anatomy. It should be noted that said central spine does not have to lie beneath the catheter in series with the suture tabs 162a-d, but might likewise perform the same purpose if located above the catheter (not shown), providing a rigid body of protection superficial to the catheter, rather than deep to the catheter or adjacent to the skin. In such an embodiment the position of the suture tabs may be different, but the general design, function and benefits of the rigid spine and ring structures will be preserved.

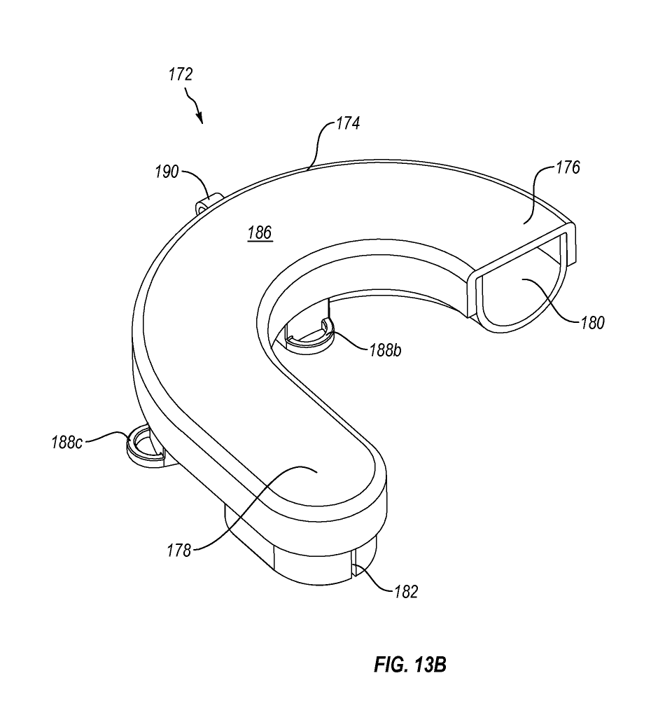

Referring now to FIGS. 13A and 13B, a catheter clamp 172 that can be closed with a catheter disposed therein is illustrated. The catheter clamp includes a curved body 174 that extends between a first end 176 and a second end 178. In the embodiment illustrated in FIG. 13A, the curved body 174 is divided into a channel portion 180 and a lid portion 186. In the embodiment illustrated in FIG. 13b, the lid portion 186 is closed over the channel portion 180. In the illustrated embodiments, the lid portion 186 is connected to the catheter clamp 172 by a tab 190. However, in some embodiment, the lid portion 186 may be separate from the catheter clamp 172.

In the open configuration shown in FIG. 13A, a catheter that has been inserted into a patient's body can be inserted into the channel 180 through the slit 182 adjacent to the second end 178. Once inserted, the catheter can be run along the curved channel 180 with the free end extending out of the first end 176 of the body 174. With the lid 186 in the closed position, the catheter clamp forms a substantially closed channel between the first 176 and second 178 ends. The channel 180 is substantially closed between the first 176 and second 178 ends, but the catheter clamp 172 includes an open portion 184 that can be positioned over the insertion site. With the lid 186 in the closed position, the channel 180 can support the curvature of the catheter and prevent kinking while the lid 186 and the opening 184 substantially (or even completely) cover the site where the catheter enters the body. The catheter clamp 172 can be affixed to the patient's body via the suture eyelets (e.g., 188b and 188c) or by any other suitable means. The site where the catheter is inserted into the body is particularly prone to infection. The embodiment illustrated in FIGS. 13A and 13B protects the site of entry and the associated tissue and blood vessels from contamination with foreign matter, bacteria, and the like.

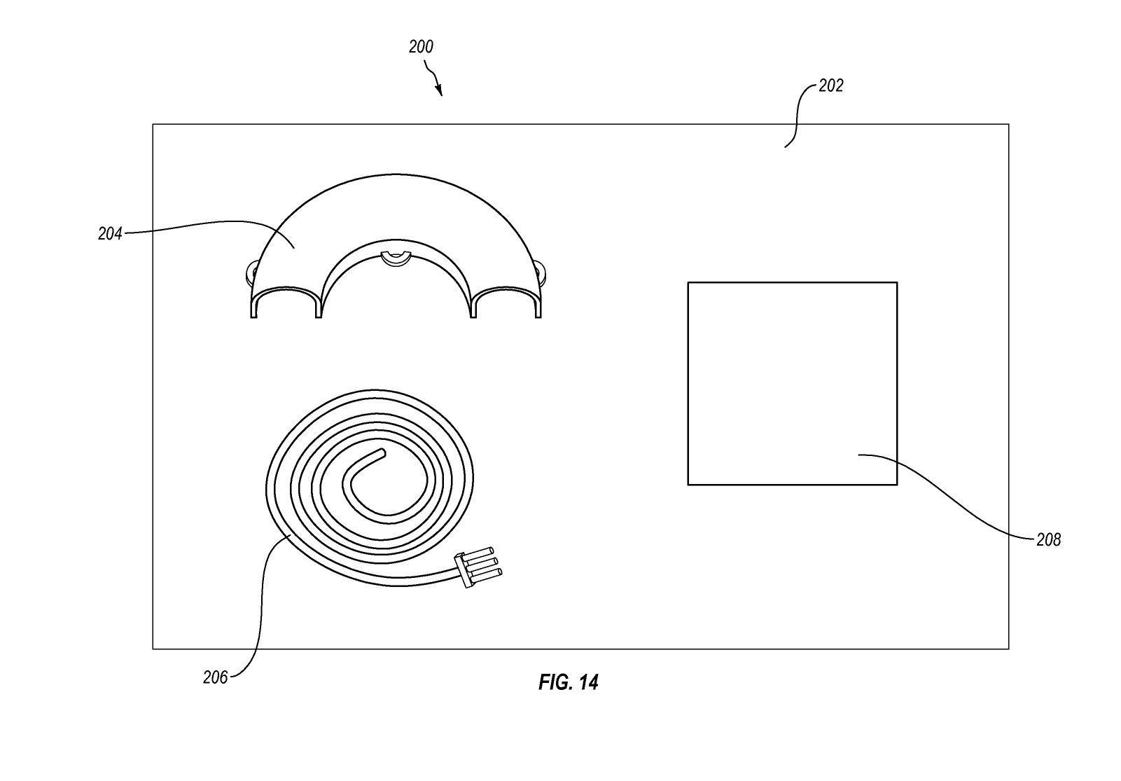

Referring now to FIG. 14, a kit 200 is described. The kit 200 may include but is not limited to, a procedure tray 202, a catheter 206, apparatus 208 for inserting the catheter into a body lumen (e.g., a vein) or a body cavity (e.g., an abdominal cavity) of a patient, and a catheter clamp 204 as described herein.

Apparatus 208 for inserting the catheter may include one or more of an anesthetic, a sterilizer for the patient's skin, a needle and suture or staples, a guide wire, a hollow or ‘cook’ needle for central venous puncture, a dilator, and a scalpel. Apparatus 208 for inserting the catheter may further include a suture needle and a length of suture, a surgical stapler, an adhesive, and/or one or more other suitable mechanisms for affixing the catheter clamp 204 to the patient's skin.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A catheter clamp, comprising:
a curved body extending between a first end and a second end;
a channel formed in the curved body into which a catheter tube can be inserted; and
wherein the curved body is shaped to permit a free end of the catheter tube to be oriented away from a catheter insertion site and to support a bent profile of the catheter tube to reduce risk of kinking the catheter tube.

2. The catheter clamp of claim 1, wherein the curved body defines an angle in a range between about 90° and about 210° to orient the free end of the catheter tube away from a catheter insertion site.

3. The catheter clamp of claim 1, wherein the curved body defines a channel with a radius of about 1.5 to about 5 cm.

4. The catheter clamp of claim 1, wherein the channel is configured to accommodate a particularly sized catheter to hold the catheter within the channel.

5. The catheter claim of claim 4, wherein the particular size of the catheter is in a range from 5 French to 15 French.

6. The catheter clamp of claim 4, wherein the particular size of the catheter is in a range from 7 French to 9 French.

7. The catheter clamp of claim 1, wherein the curved body includes a pliable material in the channel configured to accommodate and retain catheters having sizes ranging from about 5 French to about 15 French.

8. The catheter clamp of claim 1, further comprising an antimicrobial material.

9. The catheter clamp of claim 1, further comprising at least one eyelet coupled to the curved body for affixing the catheter clamp to the skin.

10. The catheter clamp of claim 1, wherein the clamp is configured for securing catheters installed at an internal jugular position, a subclavian position, a femoral position, or a peripheral vein.

11. A kit, comprising:
a catheter;
an apparatus for inserting the catheter into a lumen or a body cavity of a patient; and
the catheter clamp of claim 1.

12. The kit of claim 11, wherein the curved body defines an angle in a range between about 90° and about 210° to orient the free end of the catheter tube away from a catheter insertion site.

13. The kit of claim 11, wherein the curved body defines a channel with a radius in a range from 1.5 to 5 cm.

14. The kit of claim 11, wherein the catheter is a multi lumen catheter.

15. The kit of claim 11, wherein the apparatus for inserting the catheter includes one or more of a procedure tray, an anesthetic, a sterilizer for the patient's skin, a needle, a guide wire, a dilator, or a scalpel.

16. The kit of claim 11, further comprising a suture needle and a length of suture.

17. A method for securing a catheter to a patient's skin, the method comprising:
positioning a catheter in a body lumen or a body cavity of a patient;
positioning a free end of a catheter in the catheter clamp of claim 1; and
securing the catheter clamp to the patient's skin.

18. The method of claim 17, wherein the catheter clamp is secured to the patient's skin with at least one suture or staple passing through at least one eyelet of the clamp and into the patient's skin.

19. The method of claim 17, wherein the catheter and the catheter clamp are secured at one of a jugular position, a subclavian position, a femoral position, or a peripheral vein position.

20. The method of claim 17, wherein the size of the catheter is in a range from 5 French to 7 French and wherein the curve of the curved body of the clamp prevents kinking of the catheter in the channel.
